(19) United States
(12) Patent Application Publication
(10) Pub. No.: US 2005/0112653 A1
(43) Pub. Date: May 26, 2005

(54) Genetic archival identity card

(76) Inventors: James Rick Belcher, Tipp City, OH (US); Raymond Walter Lang, Columbus, OH (US)

Correspondence Address: Raymond Walter Lang, 3755 Olentangy Blvd., Columbus, OH, 43214, US

(21) Appl. No.: 10/973,038

(22) Filed: Oct. 25, 2004

Related U.S. Application Data
(60) Provisional application No. 60/514,787, filed on Oct. 27, 2003.

(51) Int. Cl. C12Q 1/68; C12M 1/34

(52) U.S. Cl. 435/6; 435/287.2

(57) ABSTRACT

The invention is a device for preserving genetic material for subsequent analysis. The genetic material is encapsulated into a plastic card (the device) by a process that preserves the integrity of the nucleic acid substances. The device is designed such that the genetic material can be easily recovered from the card allowing for the production of a DNA profile to aid in identification, which is the major purpose of the invention, however, the recovered nucleic acid substances can be used for many other purposes. The invention consists of the design of the plastic card device that serves to encapsulate genetic material entrapped within a matrix for secure long-term preservation.

### REFERENCE TO REALTED APPLICATION

This patent application claims the benefits of our previous US patent application Ser. No. 60/514,787 filed on Oct. 27, 2003, which is owned by the same entity as the instant application.

### OTHER REALTED REFERENCES

US Patent Documents

| | | | |
|---|---|---|---|
| 5101970 | April 1992 | Turner | 206/223 |
| 5211286 | May 1993 | Turner | 206/223 |
| 5856102 | January 1999 | Bierke-Nelson, etal | 435/6 |

Literature References
Richards B. et al. (1993) Hum. Molec. Genet. 2: 159
Ruano, G. et al. (1992) BioTechniques 13: 266
Schanke, J T and Grunerwald, H L, (1997) Epicentre Forum 4(1): 2

### STATEMENT REGARDING FEDERALLY SPONSORED RESEARCH OR DEVELOPMENT

Not Applicable

### REFERENCE TO SEQUENCE LISTING

Not Applicable

### BACKGROUND OF THE INVENTION

People have plastic identity cards of various kinds for many different purposes. The types of identity cards that have been popular in the last decade are those used for the identification of a lost or missing person, especially a child. These plastic cards can contain personal historical data, information regarding physical characteristics, sometimes a photograph and even a fingerprint. The ID card is kept in a secure place with a relative and used as a source of information to be compared, at a later date, perhaps years later, with the identity of a person found alive or in an unhappy circumstance deceased. Currently ID card devices contain only information in the form of personal data and do not incorporate genetic material containing DNA.

Many times the description of the person at the time of making the identity card, especially for children, differs significantly from the physical characteristics of the person in later years. Some times, in the case of deceased individuals, a fingerprint cannot be obtained. Almost always, however, it is possible to obtain a tissue specimen containing DNA from a person whose identity is in question. The DNA of each person is unique (except for identical twins) and contains unchanging personal genetic markers that can differentiate the individual from all other persons.

For identity purposes the ultimate identifier is one's own DNA profile. DNA profiles are the fore-most reliable method for identifying an individual in crime cases (forensic medicine). It is also used for determining genetic relationships, such as, parenthood, family trees, donor/recipient matching for transplanting tissue. Therefore, the inclusion of genetic material into a plastic card creates the best identifier device for the identification of a person. The combination of personal data and genetic material in a single device, as proposed in this invention, provides optimum utility.

Some inventors have devised systems for the banking of genetic material in a home/self storage kit for the purpose of later recovery to serve many potential purposes including identification. The samples consisting of blood, mouth cheek cells and/or hair follicles cells are placed in vials containing preservation media or in labeled envelopes and stored in a secure refrigerated environment or in a file drawer at ambient conditions. In some inventions the blood samples are dried on a filter paper card and the mouth cheek cells are dried on a collection brush, then each specimen is placed in separate labeled envelopes, sealed and stored. Some systems require refrigeration or freezing for the preservation of the genetic material, in other like systems specimens can be stored at ambient temperature as is the case for the present invention.

The previous systems for archiving genetic material differ from the present invention in the nature and design of the device for storage of the genetic material. This present invention provides for the collection and processing of nucleated cell, the best modal, and the addition of the preparation to a matrix entrapping and enhancing preservation of the DNA. The device maintains the matrix containing the genetic material in a sealed chamber that protects against contamination or degradation due to environmental factors. The device itself is constructed of a substantial plastic in a card format that can endure considerable physical stress. While maintaining the integrity in storage and on transport, the construction of the plastic card allows for easy retrieval of the genetic material for DNA extraction and analysis.

People are accustomed to using plastic cards, such as, charge cards, drivers license, medical identification, and insurance and membership cards. They are experienced in carrying them securely and keeping track of them. The plastic card with encapsulated genetic material, the invention, is therefore a convenient way for people to preserve, safeguard, transport and store their genetic identity. Since the card can be printed with identifying information on both sides, this device can provide continuity between the sample and the personal data identifiers. The Genetic Archival Identity Card does not require special storage, such as in a freezer or refrigerator.

The invention makes available to the public an inexpensive, convenient and secure device for preserving genetic material from an individual. It can occur that existing systems using vials or paper for storage may be broken open or tom thus subjecting the genetic material to contamination, loss or degradation. The construction of this device substantially reduces the potential for loss or contamination of the genetic material resulting from mechanical shearing (tearing) or spillage. In addition, any tampering with the seal chamber would be obvious to a casual observer.

### BRIEF SUMMARY OF THE INVENTION

The invention is the improved device for secure, long-term storage of genetic material for subsequent recovery of nucleic acids, such as, DNA for analysis. The genetic material is encapsulated in a plastic card (the device) by a process that preserves the integrity of the DNA. The chamber containing the genetic material is sealed to protect it from detrimental influences of the environment. Typically the card is printed on both sides with various identifying data thus creating continuity between the genetic sample and the individual's personal identification. The archival and safeguarding of an individual's genetic material is facilitated by the convenience offered by this device.

The genetic material storage device is made of substantial plastic material that is not easily destroyed by tearing or physical force unlike previous inventions. In the sealed storage chamber of the plastic card the sample cannot be spilled or contaminated and the identifying data is inseparable from the sample unlike other storage systems. The card is easily and conveniently transported, store, and provides a simple device for maintaining the confidentiality of the individual's genetic sample. Since the card chamber is permanently sealed with a waterproof vinyl patch, tampering with the sample would be obvious to anyone, which is not true of other storage systems.

In spite of its secure storage site in the card, the genetic material can be removed easily and the DNA extracted for analysis. The main purpose of the invention is for preserving genetic material to be used for subsequent identification of an individual.

### BRIEF DESCRIPTION OF THE SEVERAL VIEWS OF THE DRAWING

Not Applicable

### DETAILED DESCRIPTION OF THE INVENTION

There are many sources for collecting genetic material for preservation, however, this invention describes one best source, which involves collection body cells. An easily accessible supply of nucleated cells can be obtained by the standard procedure for recovering buccal cells, that is, mouth cheek cells. Such a sample can be supplied to the processor for application onto an acceptable matrix which is encapsulated in the device for the production of the identity card. The Genetic Archival Identity Card will be delivered to the client with instructions for storage. The surfaces of the card are suitable for customized printing to fit the end user, such as, with personal identifying data, bar coding, fingerprinting etc. Printing personal identifying data on the card makes the data and the genetic sample inseparable.

The device is a plastic card approximately the size and shape of the typical driver's license or employee's identification card. The card has a depression sufficient in size to accommodate the matrix with entrapped genetic material. The genetic material entrapped in the matrix is deposited in the depression and then encapsulated by applying a permanent cover, such as, vinyl plastic over the depression. By this method the encapsulated genetic material is protected from environmental factors that may degrade the DNA.

Prototype Evaluation:

To validate the feasibility of the invention, its construction and design, a prototype of the invention was manufactured and tested. The following is a description of one example for the production of the device.

Collection of Specimen:

The test individuals were supplied with instructions and a kit for the collection of buccal cells (mouth cheek cells) using proven standard procedures. In the laboratory the specimens were processed to yield a preparation suitable for application to a matrix. The genetic material of an individual is found in abundance in the nucleus of body cells and provides a modal source of DNA for preservation and analysis.

Construction of Device:

A depression approximately 0.28 inches in diameter and 0.03 inches deep was milled into a 40 mil PVC plastic card stock 3.4 by 2.1 inches. The drilling was performed so as not to penetrate through the card thus preserving the opposite side of the card and thereby forming a chamber. About fifty microliters of the processed buccal cell preparation was loaded onto a filter paper chad matrix 0.25 inches in diameter and allowed to air dry for 60 minutes in a sterile hood. The chad matrix with the entrapped genetic material was placed into the chamber and the surface of the depression sealed with a 3.2 mil vinyl circular waterproof stock using permanent adhesive. The vinyl seal is of sufficiently larger dimensions than the depression so as to insure that the genetic material in the chamber is completely sealed from the exterior environment.

For construction of the prototype, the depression was drilled in plastic blank stock before printing and the card was printed before loading of the chad. The genetic material was recovered from the card by punching out the chad matrix using a sterile stainless steel punch that was used to produce the chad from the filter paper matrix. The DNA was extracted and analyzed by standard procedures.

The filter paper chad, which served as the matrix for entrapment of the genetic material, has demonstrated ability to preserve the DNA. The procedure provides a secure, long-term storage of a person's genetic material for future recovery. The feasibility of the procedure was demonstrated by recovery of genetic material from prototype cards showing the expected DNA profile for the test individuals. The process of collection, incorporation, recovery, extraction and DNA analysis has been proven reliable through repeated testing using prototype cards.

Alternative methods and variations in design will allow the basic concept of long term storage of genetic material in a plastic card with the recovery of DNA for identity analysis and still achieve the purposes and concept of the invention. For example, an improvement in production may include designing a mold to produce the plastic blank stock in such a manner that the desired depression is formed in the card during manufacture. In this way the blank stock would have the desired chamber and the milling step would be unnecessary. Other examples, are the number, shape and size of the depression, and the number and source of genetic material to be stored in a single card may vary to serve a special use while still serving the purpose of the invention.

1. a device encapsulating genetic material comprising;
a) a plastic card similar in dimensions of typical charge cards
b) a plastic card of sufficient thickness to create a chamber
c) a chamber of a size and shape to accommodate genetic material in a quantity sufficient for recovery and analysis
d) genetic material entrapped in a matrix incorporated in the chamber
e) a chamber containing genetic material permanently sealed

2. the device of claim 1 is further defined;
a) by chamber meaning one or more chambers, consisting of a depression in the plastic card milled or molded with a permanent seal or a hole through the card with a permanent seal on both sides:
b) by matrix meaning a type of filter paper entrapping the genetic material.
c) by encapsulated genetic material meaning nucleic acid substances, such as, DNA and RNA, sealed in a chamber of a plastic card.
e) by genetic material meaning from any of many sources, such as, blood, cheek cells, saliva, also cells and fluids of many other body sites.
f) by seal meaning any material capable of permanently enclosing the specimen in the chamber.

3. the device of claim 1 is an improvement over existing systems since the plastic encapsulated genetic material is;
a) protected from contamination with other samples or spillage.
b) inseparable from the identification data printed on the card.
c) protected against destruction in a wide range of ambient conditions and from many environmental factors.
d) entrapped in a matrix creating a resistant environment to microbial and biochemical degradation.
e) conveniently handled, stored, and under subject's control.
f) easily recovered from the chamber for analysis and use.
g) secure, since any attempt to access the chamber would be obvious to a casual observer.
